(19) United States
(12) Patent Application Publication
(10) Pub. No.: US 2010/0152407 A1
(43) Pub. Date: Jun. 17, 2010

(54) NOVEL COMPOUNDS

(75) Inventors: David P. Birkett, Naas (IE); Matthias Voegler, Dublin (IE); Louise Gallagher, Tubbercurry (IE)

Correspondence Address: Loctite Corporation, One Henkel Way, Rocky Hill, CT, 06067, US

(73) Assignee: Loctite (R&D) Limited, Dublin (IE)

(21) Appl. No.: 12/621,561

(22) Filed: Nov. 19, 2009

Related U.S. Application Data
(60) Provisional application No. 61/138,223, filed on Dec. 17, 2008.

(51) Int. Cl. C08G 12/00; C07D 241/04; C07D 498/20

(52) U.S. Cl. 528/229; 544/402; 546/89

(57) ABSTRACT

Ketimines, enamines and oxazolidines are moisture labile functional groups that in the presence of water undergo hydrolysis to yield free amines. Within the art such latent amines have found utility in curable compositions where it is desirable to initiate cure in the presence moisture. A number of novel polyketimines, polyenamines, polymeric oxazolidines and oxazolidines are disclosed. In particular, polymeric compounds of the above classes derived from C₆ cyclic diketones and polyamines are reported. Suitable C₆ cyclic diketones include cyclohexanediones and quinones. The invention further relates to the applications of the materials, such as in moisture cure adhesives.

### CROSS REFERENCE TO RELATED APPLICATIONS

This application claims a priority filing date under 35 U.S.C. §119(e) from U.S. Provisional Application No. 61/138,223, filed Dec. 17, 2008, the disclosure of which is hereby expressly incorporated herein by reference.

### BACKGROUND

1. Field

A number of novel compounds are disclosed herein. The materials may be categorised as polyketimines, polyenamines, polymeric oxazolidines and oxazolidines. The invention further relates to the applications of the materials discussed herein.

2. Brief Description of Related Technology

Ketimines, enamines and oxazolidines are moisture labile functional groups that in the presence of water undergo hydrolysis to yield free amines. For example, the hydrolysis of a ketimine to yield ethylamine is shown in Scheme 1. Within the art such latent amines have found utility in curable compositions where it is desirable to initiate cure in the presence moisture.

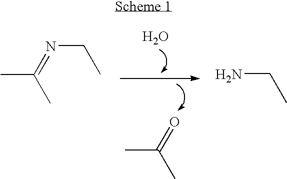

Ketimines are readily synthesised from ketones and primary amines. Enamines are obtainable from the condensation reaction of ketones/aldehydes and secondary amines. Similarly, oxazolidines are derived from ketones/aldehydes and 2-aminoethanol derivatives.

A storage-stable, one-component polyurethane composition is the subject of G.B. Patent Document No. 2290794. The composition cures at elevated temperature and comprises an isocyanate which has been blocked with a phenol or an oxime, at least one compound selected from polyketimines, polyenamines and oxazolidines, and a compound which generates water at elevated temperature. Suitable ketones disclosed for the preparation of these materials disclosed include methyl ethyl ketone, diethyl ketone, methylisobutyl ketone, diisobutyl ketone, cyclohexanones and acetophenones.

U.S. Pat. No. 3,715,338 discloses a sealing composition capable of hardening in the presence of moisture. The composition comprises a prepolymer having at least two terminal carbamic acid aryl ester groups and a polyketimine or polyenamine. Two particular polyketimines are noted: 1,6-di-(cyclohexylidene-imino)-hexane and N,N-di-(2-cyclohexylideneimino-ethyl)-cyclohexyl-(1)-amine.

U.S. Pat. No. 4,764,537 relates to an internal mold release agent for the preparation of molded articles using polyisocyanate addition polymerization comprising at least one ketimine, aldimine, enamine, cyclic Schiff base or a mixture of at least two of the above compounds. The patent discloses preparing ketimines from a myriad of ketones including cyclohexanone, 2,5-hexanedione and methylisobutylketone.

A moisture sensitive composition for the preparation of polyureas is communicated in U.S. Pat. No. 3,865,791. The composition comprises a polyisocyanate and a moisture sensitive compound that hydrolyses in the presence of water to provide an amine. The free amine initiates polymerisation of the polyisocyanate. Suitable moisture sensitive compounds listed are enamines, aldimines and ketimines. A number of ketones listed as suitable starting materials for the preparation of enamines and ketimines include cyclopentanone, cyclohexanone and substituted derivatives thereof.

U.S. Pat. No. 3,668,183 discloses polyenamine resins useful as adhesives and in coating applications. The polyenamines are produced from the reaction of polyacetoacetates or polyacetoacetamides with blocked polyamines. The polyenamines produced by the method disclosed are of the following general formula:

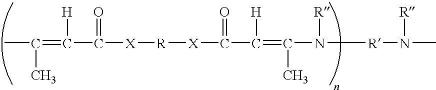

wherein R represents the base of an acetoacetate or acetoacetamide compound, X can be oxygen or nitrogen, R′ represents the base of the polyamine compound used, R″ is hydrogen or alkyl of one to six carbon atoms, and n has a value of from 1 to 3 or higher. Both R and R′ can be alkylene, cycloalkylene, arylene, arylalkylene, cycloalkylalkylene, alkylcycloalkylene, alkylarylene or any one or more of these groups linked together with etheric oxygen (—O—) moieties. R and R′ can contain from one to 50 carbon atoms, preferably from one to 15 carbon atoms.

Polyamines obtained from the hydrolysis of polyketimines and polyenamines are disclosed in U.S. Pat. No. 4,224,417. The polyamines disclosed fined utility in the preparation of polyurethane resins.

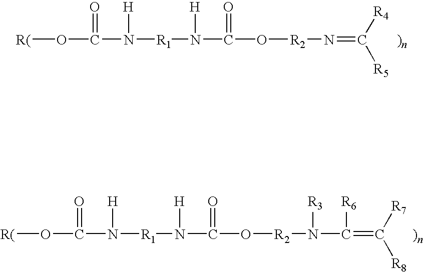

within these general formulae n represents an integer of between 2 and 10, R represents an n-valent organic group such as can be obtained by the removal of n hydroxyl groups from a polyhydroxyl compound which has an average molecular weight of between about 200 and about 10,000 and may also contain urethane groups. R1 represents an alkylene, cycloalkylene, arylene or aralkylene group containing 2 to 15 carbon atoms, R2 represents an alkylene, cycloalkylene, arylene or aralkylene group with from 2 to 15, preferably 6 to 12, carbon atoms which may contain an —O— or —N(R₃)— group and R₃ represents hydrogen, or an alkyl or cycloalkyl group with 1 to 9 carbon atoms, or an alkylene group which together with R₂ and —NH— forms a 5- or 6-membered ring. The groups R4 to R8 represent alkyl, cycloalkyl, aryl and aralkyl groups with from 1 to 15 and preferably 1 to 7 carbon atoms or R4 and R5 together or R6 and R8 together form a 5- to 12-membered ring and/or R4, R6 and/or R7 represent hydrogen atoms. This patent does not disclose polymeric ketimines and enamines according to the present invention.

U.S. Pat. No. 4,663,483 discloses the preparation of polyenamines from diacetylenic diketones and aromatic diamines.

Notwithstanding the state of the art it would be desirable to provide alternative polyketimines, polyenamines and (polymeric)oxazolidines capable of synthesis from a simple starting ketone. Such polymeric compounds would comprise a simple repeat unit comprised of readily available, inexpensive starting materials.

### SUMMARY OF THE INVENTION

The present invention provides for polymeric compounds derived from C₆ cyclic diketones and polyamines. Such compound show advantages in terms of latency or stability in the moisture cure of for example epoxies.

In one aspect the present invention provides for a polymeric compound formed from the condensation reaction between:

a cyclohexanedione, 2-cyclohexene-1,4-dione or a quinone; and

(ii) a polyamine.

As will be appreciated by a person skilled in the art cyclohexanediones, 2-cyclohexene-1,4-dione and quinones are C₆ cyclic diketones readily available from commercial suppliers such as Sigma Aldrich.

The polymeric compound of the present invention may be formed from the condensation reaction between a cyclohexanedione and a polyamine, wherein the cyclohexanedione is selected from 1,2-cyclohexanedione, 1,3-cyclohexanedione, 1,4-cyclohexanedione and combinations thereof. Desirably, the cyclohexanedione is 1,4-cyclohexanedione. Cyclohexanediones are inexpensive starting materials and are readily available.

Preferably, the polymeric compound of the present invention is formed from the condensation reaction between a quinone and a polyamine, wherein the quinone may be selected from the group consisting of 1,2-quinone, 1,4-quinone and combinations thereof. Quinones provide a basic structural element upon which a conducting polymer can be synthesised.

Desirably, the polyamine is commercially available and may be selected from primary diamines, secondary diamines, polyethyleneimine, polyoxyalkyleneamines, tris(2-aminoethyl)amine, amine terminated butadiene acrylonitrile (ATBN) and combinations thereof. In one embodiment, the polyamine may be a primary diamine. In a further embodiment, the polyamine may be a secondary diamine. The term “primary diamine” refers to a diamine having two primary amine groups. Similarly, the term “secondary diamine” refers to a diamine having two secondary amine groups.

Within the art, the term “polyoxyalkyleneamines” is sometimes used interchangeably with “polyetheramines”. Exemplary materials include those available under the trade name Jeffamine®. The polyether backbone is normally based on either propylene oxide, ethylene oxide, or a combination thereof. Exemplary primary and secondary Jeffamine® diamines are shown below, wherein x can be 2 to 70. However, it will be appreciated that the particular Jeffamines® illustrated below serve only as examples and are in no way to be interpreted as limiting.

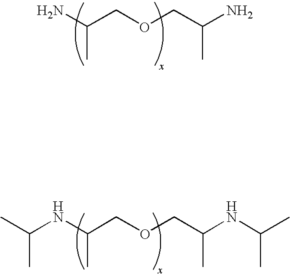

Desirably, the primary diamine is selected from the group consisting of 4-aminoaniline, cyclohexanediamines, cyclopentane diamines, hexamethylene diamine, xylenediamines, diaminonapthalenes, 2,2′-oxydiethylamine, polyoxyalkylene primary diamines, and combinations thereof. Such primary diamines are commercially available.

Desirably, the secondary diamine is selected from the group consisting of piperazine, dipiperidines, polyoxyalkylene secondary diamines and combinations thereof. Further desirably, the dipiperidine is 4,4′-trimethylene dipiperidine. Such secondary diamines are commercially available.

In one embodiment, the present invention provides for a polyketimine (or polymeric ketimine). Desirably, the polyketimine is derived from a cyclohexanedione and is of the general formula:

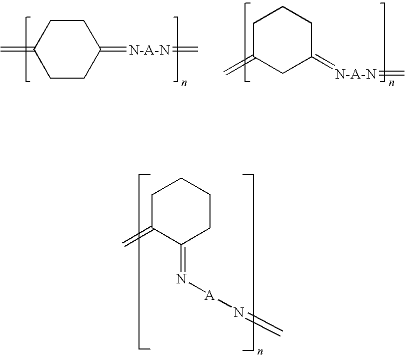

wherein,
n is 2 to 100, and
A comprises C₁-C₂₀ alkyl, C₁-C₂₀ cycloalkyl, C₆-C₂₀ aromatic and combinations thereof.

In one embodiment A comprises a cyclohexyl ring. In a further embodiment, A comprises a phenyl group. In the embodiment where A is a phenyl group, the invention provides for a polyketimine comprising a C₆ alkyl-N—C₆ aromatic repeat unit, as shown below:

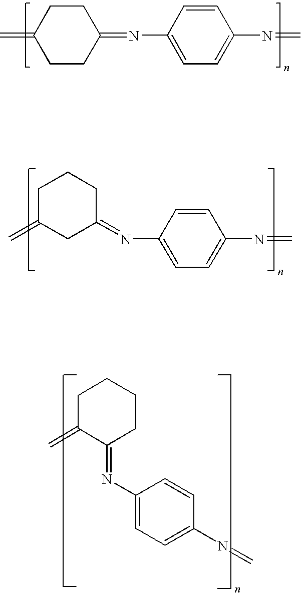

In a further embodiment, the present invention provides for polyenamine (or polymeric enamine). Desirably, the polyenamine is derived from a cyclohexanedione and is of the general formula:

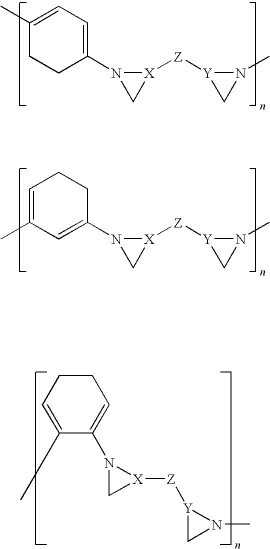

wherein,
n is 2 to 100,
X and Y can be the same or different and can be C₁-C₁₁; and
Z can be C₁-C₂₀ alkyl, C₁-C₂₀ cycloalkyl, C₆-C₂₀ aromatic and combinations thereof.

Desirably, X and Y are the same and comprise C₃-C₄. Further desirably, Z comprises C₁-C₅ alkyl. In a one embodiment X and Y comprise C₄. In a further embodiment Z comprises C₃ alkyl. In a preferred embodiment X and Y comprise C₄ and Z comprises C₃ alkyl. In this embodiment the secondary diamine comprises 4,4′-trimethylenedipiperidine.

The invention further provides for a polyenamine of the general formula:

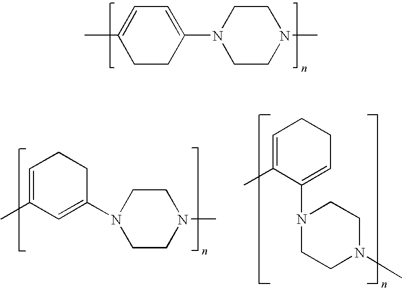

wherein,
n is 2 to 100.

In a further embodiment the present invention provides for a polymer having mixed enamine and ketimine functionality. Such a polymer is formed from the condensation reaction of:

(i) a cyclohexanedione, 2-cyclohexene-1,4-dione or a quinone; and

(ii) a polyamine having primary and secondary amine groups; and/or

(iii) a primary diamine and a secondary diamine.

Desirably, the polymer is formed from the condensation reaction of a cyclohexanedione and a polyamine having primary and secondary amine groups, wherein the cyclohexanedione is selected from the group consisting of 1,2-cyclohexanedione, 1,3-cyclohexanedione, 1,4-cyclohexanedione and combinations thereof. In a further embodiment, the polymer is formed from the condensation reaction of a quinone and a polyamine having primary and secondary amine groups, wherein the quinone may be selected from the group consisting of 1,2-quinone, 1,4-quinone and combinations thereof. In one embodiment the polyamine having primary and secondary amine groups comprises polyethyleneimine. In a further embodiment the polyamine having primary and secondary amine groups is a diamine with one primary amine and one secondary amine. Suitable diamines having primary and secondary amine groups include 2-(1-piperazinyl)ethanamine (below).

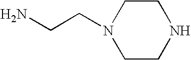

In yet a further embodiment, the present invention provides for a (conducting) polyketimine, derived from 2-cyclohexene-1,4-dione or a quinone, of the general formula:

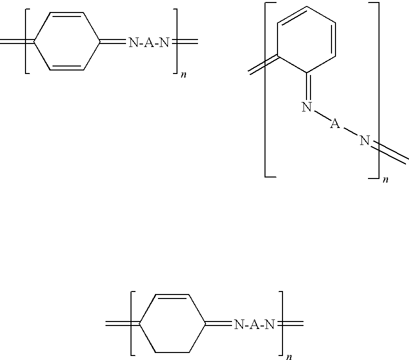

wherein,
n is 2 to 100, and
A comprises a C₆-C₂₀ aromatic, a phenyl group, a naphthyl group and combinations thereof.

In one embodiment, A comprises a C₆ aromatic ring. Polyketimines comprising non-interrupted conjugation of the π-electron system in the polymer are suitable for use as conducting polymers.

Similarly, the invention provides for a (conducting) polyenamine selected from:

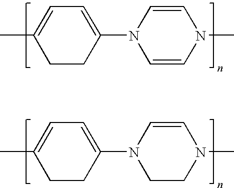

wherein,
n is 2 to 100.

Polyenamines comprising non-interrupted conjugation of the π-electron system in the polymer are suitable for use as conducting polymers.

In a further aspect the present invention provides for an assembly comprising a conductive pathway formed from a conductive polyenamine or conducting polyketimine of the present invention. Desirably, the conductive pathway is formed from a conducting polyketimine according to the present invention. Such assemblies may find use in electrochromic devices, semi-conducting devices and the like.

As will be appreciated by a person skilled in the art, the cyclohexyldiene ring of the polyenamines of the present invention can undergo oxidation to the corresponding phenyl ring, for example (wherein n is 2 to 100):

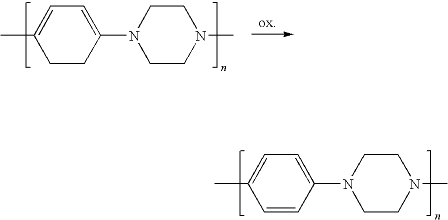

As such, the present invention extends to a polyenamine wherein the cyclohexyldiene ring of the polyenamine repeat unit is oxidised to a phenyl ring.

In a further aspect the invention provides for a method of preparing a polyketimine or polyenamine according to the present invention comprising the steps of:
(i) providing a reaction mixture of:
   a. a cyclohexanedione, 2-cyclohexene-1,4-dione or a quinone; and
   b. a polyamine;
(ii) heating the reaction mixture to generate a condensation product and water; and
(iii) removing the water or the condensation product from the reaction mixture as it is generated.

Desirably, water is removed from the reaction mixture as it is formed. This is readily achieved by azeotropic removal.

In yet a further aspect the present invention provides for a one-part moisture cure adhesive composition comprising:
a curable monomer; and
(ii) a polyketimine according to the present invention,
wherein moisture generates an amine from component (ii) thereby initiating cure of the curable monomer.

Desirably, polymeric ketimines suitable for use in a one-part moisture cure adhesive composition do not comprise non-interrupted conjugation of the π-electron system in the polymeric ketimine. Such a conjugated π-electron system in the polymer will increase the resistance of the polymer to hydrolysis.

In one embodiment, the curable monomer comprises at least one functional group selected from epoxy, urethane and combinations thereof. Desirably, the curable monomer comprises at least one epoxy functional group.

Polyketimines of the present invention show advantages in terms of latency or stability in moisture cure compositions according to the present invention.

In another aspect, the present invention provides for a polymeric oxazolidine formed from the condensation reaction between:

(a) a cyclohexanedione, 2-cyclohexene-1,4-dione or a quinone; and

(b) a bis(2-aminoethanol).

In one embodiment, the polymeric oxazolidine is formed from the condensation reaction of a cyclohexanedione and a bis(2-aminoethanol), wherein the cyclohexanedione is selected from the group consisting of 1,2-cyclohexanedione, 1,3-cyclohexanedione, 1,4-cyclohexanedione and combinations thereof. Desirably, the cyclohexanedione is 1,4-cyclohexanedione. In a further embodiment, the polymer is formed from the condensation reaction of a quinone and a bis(2-aminoethanol), wherein the quinone may be selected from 1,2-quinone, 1,4-quinone and combinations thereof.

As used herein the term bis(2-aminoethanol) refers to compounds of the general formula:

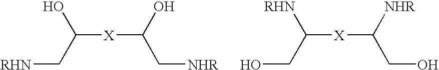

wherein X can be C₁-C₂₀ alkyl, C₁-C₂₀ cycloalkyl, C₆-C₂₀ aromatic and combinations thereof; and R can be C₁-C₂₀ alkyl, C₁-C₂₀ cycloalkyl, C₆-C₂₀ aromatic and combinations thereof.

In one embodiment, X is C₁-C₆ alkyl.

In a preferred embodiment, the polymeric oxazolidine of the present invention is of the general formula:

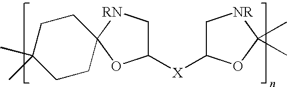

wherein n is 2 to 100,
X can be C₁-C₂₀ alkyl, C₁-C₂₀ cycloalkyl, C₆-C₂₀ aromatic and combinations thereof; and
R can be C₁-C₂₀ alkyl, C₁-C₂₀ cycloalkyl, C₆-C₂₀ aromatic and combinations thereof.

Desirably, X is C₁-C₆ alkyl.

The invention further provides for an oxazolidine of the general formula:

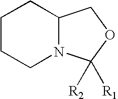

wherein R1 and R2 can be the same or different and are selected from C₁-C₂₀ alkyl, C₃-C₂₀ cycloalkyl C₅-C₂₀ aryl, and C₆-C₂₀ alkylaryl and wherein R1 and R2 may comprise a C₃-C₂₀ cycloalkyl moiety.

In one embodiment, R1 and R2 are selected from methyl, ethyl and iso-butyl.

In a further aspect the invention provides for an ketimine of the general formula:

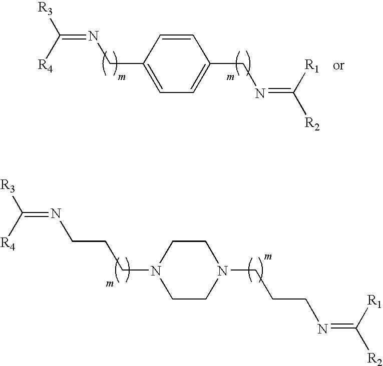

wherein
m is 0 to 20;
R1, R2, R3 and R4 can be the same or different, and are selected from C₁-C₂₀ alkyl, C₅-C₂₀ aryl, and C₆-C₂₀ alkylaryl;
wherein R1 and R2 may comprise a C₃-C₂₀ cycloalkyl moiety; and
R3 and R4 may comprise a C₃-C₂₀ cycloalkyl moiety

In one embodiment, R1, R2, R3 and R4 are selected from methyl, ethyl and isobutyl. Preferably, R1 and R2 and or R3 and R4 comprise a cyclohexyl ring.

The invention further relates to a one-part moisture cure adhesive composition comprising:
(i) a curable monomer capable of curing in the presence of an amine; and
(ii) a polymeric oxazolidine or oxazolidine according to the present invention; or
(iii) a ketimine according to the present invention (see paragraph [0046]), wherein water generates an amine from component (ii) or (iii) thereby initiating cure of the curable monomer.

In one embodiment, the curable monomer comprises at least one functional group selected from epoxy, urethane and combinations thereof. In a further embodiment, the curable monomer comprises at least one epoxy functional group.

Polymeric oxazolidines and oxazolidines of the present invention show advantages in terms of latency or stability in moisture cure compositions according to the present invention.

In a further aspect the present invention provides for a polymer having mixed oxazolidine, enamine and/or ketimine functionality. Such a polymer is formed from the condensation reaction of:

(i) a cyclohexanedione, 2-cyclohexene-1,4-dione or a quinone;

(ii) a polyamine having primary and/or secondary amine groups; and/or

(iii) a primary diamine and/or a secondary diamine; and

(iv) a bis(2-aminoethanol) as defined above.

In one embodiment, the polymer is formed from the condensation reaction of a cyclohexanedione selected from 1,2-cyclohexanedione, 1,3-cyclohexanedione, 1,4-cyclohexanedione and combinations thereof. Desirably, the cyclohexanedione is 1,4-cyclohexanedione. In a further embodiment, the polymer is formed from the condensation reaction of a quinone selected from 1,2-quinone, 1,4-quinone and combinations thereof.

In one embodiment the polyamine having primary and secondary amine groups comprises polyethyleneimine. In a further embodiment the polyamine having primary and secondary amine groups is a diamine with one primary amine and one secondary amine. Suitable diamines having primary and secondary amine groups include 2-(1-piperazinyl)ethanamine.

The invention further provides for a one-part moisture cure adhesive composition comprising:
(i) a curable monomer capable of curing in the presence of an amine; and
(ii) a polymer having mixed oxazolidine, enamine and/or ketimine functionality according to the present invention,
   wherein water generates an amine from component (ii) thereby initiating cure of the curable monomer.

In one embodiment, the curable monomer comprises at least one functional group selected from epoxy, urethane and combinations thereof. In a further embodiment, the curable monomer comprises at least one epoxy functional group.

Where suitable, it will be appreciated that all optional and/or preferred features of a one embodiment of the invention may be combined with optional and/or preferred features of another/other embodiment(s) of the invention.

### DETAILED DESCRIPTION OF THE INVENTION

Additional features and advantages of the present invention are described in, and will be apparent from, the detailed description of the invention:

### Synthesis of Polymeric Enamines

Several combinations of difunctional ketones and secondary diamines were tested for synthesis of polymeric enamines. Reactants mixed in a molar ration of 1:1 and refluxed in toluene with azeotropic removal of water. Solid precipitates are formed after 6-10 h. Products show generally poor solubility in common organic solvents. Analysis is therefore largely restricted to IR (KBr pellets).

### 1,4-Cyclohexanedione & Piperazine

Product: Dark brown precipitate

IR analysis: presence of C═C, C═C—H

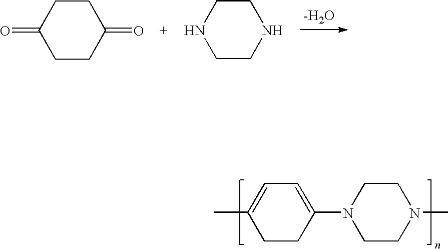

wherein n is 2-100

### Synthesis of Polymeric Imines

Several combinations of difunctional ketones and primary diamines were tested for synthesis of polymeric imines. Reactants mixed in a molar ration of 1:1 and refluxed in toluene with azeotropic removal of water. Solid precipitates are formed after 6-10 h. Products show generally poor solubility in common organic solvents. Analysis is therefore largely restricted to IR (KBr pellets).

### 1,4-Cyclohexanedione & trans-1,4-Cyclohexanediamine

Product: Dark brown precipitate

IR analysis: 1693, 1653 C═N

1584, 1508 C═C

Suggested Structure: Mix of two Ketimines. Intense brown colour of product and presence of C═C bands suggest partial oxidation into the corresponding quinone-imine.

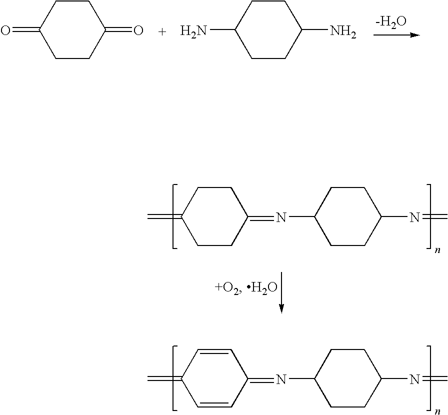

wherein n is 2-100

### 1,4-Cyclohexandione with p-Phenylenediamine in N₂

Product: light beige solid. Product turns to dark brown and black within 1-2 days upon exposure to air

IR analysis: 1706 C═O (starting material)

1647-1608 multiple C═N

1504 C═C (aromatic)

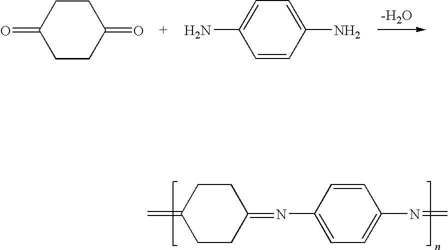

wherein n is 2-100
Suggested Structure: IR shows some remaining starting material (ketone) despite long reaction time (15 h). Broad C═N band suggests formation of several adducts of stoichiometries other than 1:1. No significant change in IR upon exposure to air (monitored over 6 h).

### 1,4-Cyclohexandione with p-Phenylenediamine

Product: Black insoluble solid formed

IR analysis: 1706 C═O (starting material)

1647-1608 multiple C═N

1504 C═C (aromatic)

IR shows some remaining starting material (ketone) despite long reaction time (15 h). Broad C═N band suggests formation of several adducts of stoichiometries other than 1:1. Dark colour of product might indicate partial oxidation forming trace amounts of quinoid form. However, the absence of aliphatic C═C bands in the IR precludes formation of significant amounts of the quinone-imine.

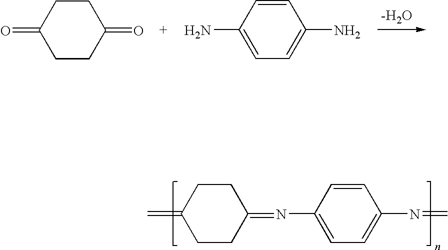

wherein n is 2-100

### Synthesis of Conducting Polyketimines

### p-Benzoquinone with p-Phenylenediamine

Product: Black insoluble solid formed

IR analysis: 1625 C═N

1564 C═C (aliphatic)

1512, 1482 C═C (aromatic)

### Suggested Structure:

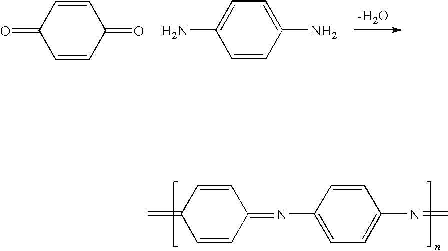

wherein n is 2-100

### Oxazolidine Synthesis

General Schematic

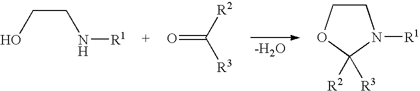

Reactions of Amines/Aminoalcohols with Methyl-isobutyl-ketone or Cyclohexanone
The amine or aminoalcohol (ca. 20 g) was dissolved in 100 ml anhydrous toluene. A total of 3 molar equivalents (6 eq. for difunctional amines) of the ketone (MIBK or Cyclohexanone) are added and the solution is then refluxed for ca. 8-12 h with azeotropic removal of water in a Dean-Stark trap. After completion of the reaction (no more water formation observed), the solvent and all volatiles are removed in vacuo and the remaining crude product is purified by vacuum distillation.

### 1,4-Bis(3-aminopropyl)piperazine+MIBK

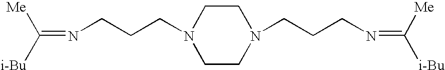

light orange oil

Yield=49%

b.p.=150° C./20 mbar

¹H-NMR (CDCl₃) δ (ppm) 3.28 (t, 4H, C═N—CH₂CH₂CH₂—N)

2.47 (s, 8H, N—CH₂—CH₂—N)

2.5-2.0 (m, 12H)

1.79 (s, 6H, N═C—CH₃)

0.91 (d, 12H CH—(CH₃)₂)

IR (cm⁻¹): v (C—H)=2950, 2869, 2805, 2768

v (C═N) 1657 (st)

MS (m/z): 364 [M⁺], 169

### 2-Piperidinemethanol & MIBK

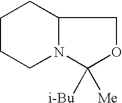

White crystalline solid

b.p.=120° C./12 mbar

yield=35%

two diastereomers identified in GC-MS (ratio ca. 4:1),
spectroscopic data given for main isomer

¹H-NMR (CDCl₃): δ (ppm) 3.49 (d, 2H, CH₂O)

2.62 (m, 3H, CH₂N, CHN)

1.46 (m, 8H, —CH₂—)

1.35 (s, 2H, —CH₃)

1.00 (d, 6H, —CH₃)

IR (cm⁻¹): v (C—H)=2929, 2858

v (O—C—N)=1075, 1023 (m)

MS (m/z) main isomer: 197 [M⁺], 182 [M⁺-CH₃], 140

Reactions of Amines/Aminoalcohols with Acetone

The amine/aminoalcohol (ca. 20 g) is dissolved in ca. 100 ml of dry acetone. The solution is heated to ca. 60° C. and the acetone/water azeotrope distilled off completely. The remaining residue is then redissolved in dry acetone and the procedure is repeated for completion of the reaction. All volatiles are removed from the reaction mixture and the remaining crude product purified by vacuum distillation

p-Xylenediamine & Acetone

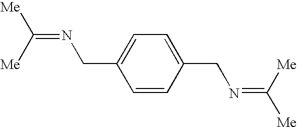

yield=21.6%

light yellow solid,

b.p. 120° C./0.2 mbar

¹H-NMR (CDCl₃): δ (ppm) 7.21 (s, 4H, Ar—H)

4.41 (s, 4H, CH₂N)

2.06 (s, 6H, CH₃)

1.90 (s, 6H, CH₃)

IR (cm⁻¹): v (C—H)=3285 (w), 2909 (m), 2855 (m)

v (C═N)=1660 (s)

MS (m/z) 216 [M⁺-H], 160, 144 [Me₂C═NCH₂Ph⁺]

### 2-Piperidinemethanol & Cyclohexanone

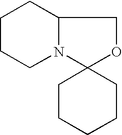

Clear oily liquid

b.p.=90° C./2 mbar

yield=83%

¹H-NMR (CDCl₃): δ (ppm) 3.88 (d, 2H, CH₂O)

3.33 (t, 2H, CH₂N)

1.54 (m, 16H, —CH₂—)

IR (cm⁻¹): v (C—H)=2928, 2858, 2797

v (OCN), 1074, 1036, 1024 (m)

MS (m/z) 195 [M⁺], 166 [M⁺-CHO], 97 [C₅H₉NCH₂⁺], 82 [C₆H₁₀⁺]

The words “comprises/comprising” and the words “having/including” when used herein with reference to the present invention are used to specify the presence of stated features, integers, steps or components but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A polymeric compound formed from the condensation reaction between:
(i) a cyclohexanedione, 2-cyclohexene-1,4-dione or a quinone; and
(ii) a polyamine.

2. A polymeric compound according to claim 1 formed from the condensation reaction between a cyclohexanedione and a polyamine, wherein the cyclohexanedione is selected from the group consisting of 1,2-cyclohexanedione, 1,3-cyclohexanedione, 1,4-cyclohexanedione and combinations thereof.

3. A polymeric compound according to claim 1 formed from the condensation reaction between a quinone and a polyamine, wherein the quinone is selected from the group consisting of 1,2-quinone, 1,4-quinone and combinations thereof.

4. A polymeric compound according to claim 1 wherein the polyamine is selected from the group consisting of primary diamines, secondary diamines, polyethyleneimine, polyoxyalkyleneamines, tris(2-aminoethyl)amine, amine terminated butadiene acrylonitrile and combinations thereof.

5. A polymeric compound according to claim 1, wherein the polyamine comprises a primary diamine and is selected from the group consisting of 4-aminoaniline, cyclohexanediamines, cyclopentane diamines, hexamethylene diamine, xylenediamines, diaminonapthalenes, 2,2′-oxydiethylamine, polyoxyalkylene primary diamines, and combinations thereof.

6. A polymeric compound according to claim 1, wherein polyamine comprises a secondary diamine and is selected from the group consisting of piperazine, dipiperidines, polyoxyalkylene secondary diamines and combinations thereof.

7. A polymeric compound according to claim 1 wherein the polymer comprises a polyketimine.

8. A polymeric compound according to claim 7 wherein the polyketimine is of the general formula: 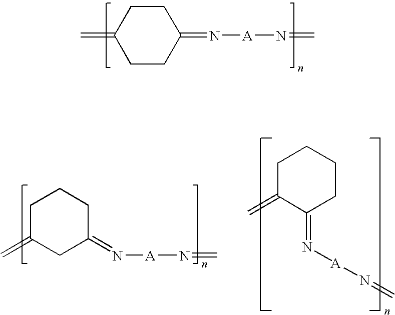
wherein,
n is 2 to 100, and
A comprises C₁-C₂₀ alkyl, C₁-C₂₀ cycloalkyl, C₆-C₂₀ aromatic and combinations thereof.

9. A polymeric compound according to claim 8 wherein A comprises a cyclohexyl ring or a phenyl group.

10. A polymeric compound according to claim 7 wherein the polyketimine is of the general formula: 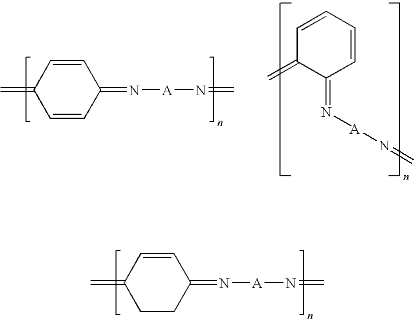
wherein,
n is 2 to 100, and
A comprises a C₆-C₂₀ aromatic, a phenyl group, a naphthyl group and combinations thereof.

11. A polymeric compound according to claim 10 wherein A comprises a C₆ aromatic ring.

12. A polymeric compound according to claim 1 wherein the polymer comprises a polyenamine.

13. A polymeric compound according to claim 12 wherein the polyenamines is of the general formula: 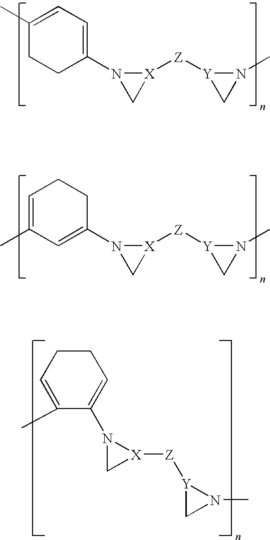
wherein,
n is 2 to 100,
X and Y can be the same or different and can be C₁-C₁₁; and
Z can be C₁-C₂₀ alkyl, C₁-C₂₀ cycloalkyl, C₆-C₂₀ aromatic and combinations thereof.

14. A polymeric compound according to claim 13 wherein X and Y comprise C₄ and Z comprises C₃ alkyl.

15. A polymeric compound according to claim 12 wherein the polyenamine is of the general formula: 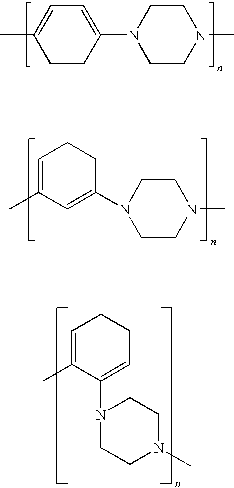
wherein,
n is 2 to 100.

16. A polymeric compound according to claim 12 wherein the polyenamine is of the general formula: 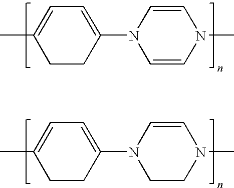
wherein,
n is 2 to 100.

17. A polymeric compound according to claim 1 having mixed enamine and ketimine functionality.

18. A polymeric oxazolidine formed from the condensation reaction between:
(a) a cyclohexanedione, 2-cyclohexene-1,4-dione or a quinone; and
(b) a bis(2-aminoethanol).

19. A polymer having mixed oxazolidine, enamine and/or ketimine functionality formed from the condensation reaction of:
(i) a cyclohexanedione, 2-cyclohexene-1,4-dione or a quinone;
(ii) a polyamine having primary and/or secondary amine groups; and/or
(i) a primary diamine and/or a secondary diamine; and
(ii) a bis(2-aminoethanol).

20. A polymeric oxazolidine according to claim 19 wherein the bis(2-aminoethanol) is of the general formula: 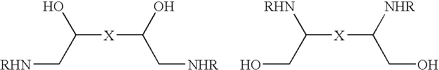
wherein X can be C₁-C₂₀ alkyl, C₁-C₂₀ cycloalkyl, C₆-C₂₀ aromatic and combinations thereof; and
R can be C₁-C₂₀ alkyl, C₁-C₂₀ cycloalkyl, C₆-C₂₀ aromatic and combinations thereof.
